(19) United States
(12) Patent Application Publication
(10) Pub. No.: US 2026/0158251 A1
(43) Pub. Date: Jun. 11, 2026

(54) RF MICRONEEDLING WITH ADJUSTABLE NEEDLE-TO-TISSUE POWER COUPLING

(71) Applicant: CYNOSURE, LLC, Westford, MA (US)

(72) Inventors: Mirko Mirkov, Chelmsford, MA (US); James Boll, Montclair, NJ (US); Timothy Harrah, Cambridge, MA (US)

(73) Assignee: CYNOSURE, LLC, Westford, MA (US)

(21) Appl. No.: 19/390,171

(22) Filed: Nov. 14, 2025

Related U.S. Application Data
(60) Provisional application No. 63/728,550, filed on Dec. 5, 2024.

(51) Int. Cl. A61M 37/00; A61B 18/00; A61B 18/14

(52) U.S. Cl. CPC A61M 37/00; A61B 18/14; A61B 2018/0047; A61B 2018/1467; A61M 2037/003; A61M 2037/0061; A61M 2202/0007; A61M 2202/0468

(57) ABSTRACT

In part, in one embodiment, the disclosure relates to an apparatus for tissue treatment. The apparatus includes a plurality of hollow needles attached to the base, wherein each needle defines a hollow portion, at least one of the hollow portions of the plurality of hollow needles is in fluid communication with one or more fluid sources for delivering an electrically conductive fluid; a guard that is movably attached to the base; and an electrical interface configured to electrically couple the plurality of hollow needles to a control system, the electrical interface configured to receive one or more control signals, the control signal configured to managing at least one of (i) the temperature of fluid prior to delivery into at least one of the hollow portions and (ii) the fluid delivered into at least one of the hollow portions.

### CROSS REFERENCE TO RELATED APPLICATIONS

This application claims priority to and the benefit of U.S. Provisional Application No. 63/728,550 filed on Dec. 5, 2024, the entire disclosure of which is incorporated by reference herein.

### FIELD

This disclosure relates generally to the field of microneedling and tissue treatment.

### BACKGROUND

Electrosurgical devices are known for applying RF energy to tissue so as to generate a variety of effects, including invasive procedures (e.g., for ablating or vaporizing tissue) or less-invasive procedures (e.g., to heat the surface of the skin). However, a need remains for improved methods and systems for providing RF energy in cosmetic and/or aesthetic applications, for example, in order to improve the appearance of skin for example, so that it is (or appears) tightened/smoothed.

### SUMMARY

In part, in one aspect, the disclosure relates to an apparatus for tissue treatment. The apparatus includes a plurality of hollow needles attached to the base, wherein each needle defines a hollow portion, at least one of the hollow portions of the plurality of hollow needles is in fluid communication with one or more fluid sources for delivering an electrically conductive fluid; a guard that is movably attached or slidably disposed relative to the base; and an electrical interface configured to electrically couple the plurality of hollow needles to a control system, the electrical interface configured to receive one or more control signals from the control system, the control signal configured to managing at least one of (i) the temperature of fluid prior to delivery into at least one of the hollow portions and (ii) the volume of fluid delivered into at least one of the hollow portions.

In various embodiments, the electrically conductive fluid is configured to modify electrical conductivity of tissue. In many embodiments, the guard is configured to protect the plurality of hollow needles, wherein the guard is configured to attach to a handpiece. In various embodiments, a portion of a surface of each needle comprises an electrically insulative coating. In many embodiments, the apparatus further includes an actuator in mechanical communication with the plurality of hollow needles. In many embodiments, the apparatus further includes microfluidic channels that align with at least one of the hollow portions of the plurality of hollow needles providing fluid communication with one of more fluid sources for delivering an electrically conductive fluid. In various embodiments, the electrically conductive fluid does not have analgesic properties. In many embodiments, the electrically conductive fluid causes biochemical changes to the tissue. In many embodiments, the apparatus further includes a microfluidic assembly, the microfluidic assembly defining a set of microfluidic channels that align with at least one of the hollow portions of the plurality of hollow needles to deliver the electrically conductive fluid therethrough and to an outlet of each needle.

In various embodiments, the electrically conductive fluid comprises one or more of a gas, a liquid, and a gel. In many embodiments, controlling the temperature of the fluid determines whether the fluid comprises one or more of a gas, a liquid, and a gel. In various embodiments, the fluid thermally modifies a portion of the tissue or a tissue structure. In some embodiments, the thermal modification to tissue can be one or more of denaturation, apoptosis, stimulation, coagulation, necrosis, charring, and ablation. In various embodiments, the fluid modifies a tissue structure or a portion of tissue or a tissue region. In many embodiments, the apparatus further includes an energy source provided in communication with at least one of the needles. In many embodiments, the apparatus further includes a control system coupled to the electrical interface, the control system comprising a control module that varies characteristics of energy supplied by the energy source. In various embodiments, more than one of the needles are electrically conductive, and, wherein the energy source is configured to supply radio frequency electromagnetic energy to individual ones of the needles. In various embodiments, the radio frequency electromagnetic energy is delivered in a bipolar mode. In various embodiments, the radio frequency electromagnetic energy is delivered in a monopolar mode. In some embodiments, the energy is configured to deliver thermal energy or RF energy relative to a tissue region.

In many embodiments, the apparatus further includes balancing control of the electrical conductivity of the fluid to enable use of a relatively lower RF nominal power level and a relatively narrower RF impedance range than would be needed in the absence of balancing control of the electrical conductivity. In many embodiments, the apparatus further includes balancing control of the temperature of the fluid prior to flowing into at least one of the hollow portions to enable use of a relatively lower RF nominal power level and a relatively narrower RF impedance range than would be needed in the absence of balancing control of fluid temperature.

In many embodiments, the apparatus further includes balancing control of at least one of (a) the electrical conductivity of the fluid and (b) the temperature of the fluid prior to fluid flowing into at least one of the hollow portions to enable use of a relatively lower RF nominal power level and a relatively narrower RF impedance range than would be needed in the absence of balancing control of these.

In various embodiments, one or more of the plurality of hollow needles has a cut angle that ranges from about 20° to about 70°. In various embodiments, the selected cut angle determines how a given amount of fluid distributes about the needle tip to be in contact with tissue adjacent the needle tip. In many embodiments, the apparatus further includes a handpiece configured to attach to and detach from the base. In some embodiments, the handpiece comprises at least one reservoir in fluid communication with at least one of the plurality of hollow needles. In various embodiments, the at least one reservoir comprises saline solution. In some embodiments, the at least one reservoir comprises a deformable container configured to deliver reservoir contents into at least one of the hollow portions of the plurality of hollow needles in response to manual activation or a control signal from a control system. In various embodiments, the reservoir contents include an electrically conductive saline solution.

In part, in another aspect, the disclosure relates to a skin treatment method. The method includes inserting a plurality of hollow needles into a dermal layer of skin, the plurality of hollow needles being attached to a base; inducing a pattern of fractional tissue modification into a target tissue by delivering an electrically conductive fluid to the target tissue through two or more of the hollow portions of plurality of hollow needles; controlling at least one of (i) the temperature of an electrically conductive fluid prior to delivery into two or more of the hollow portions; (ii) the electrical conductivity of an electrically conductive fluid delivered into two or more of the hollow portions; and (iii) an electrical parameter measured by a control system in response to delivery of an electrical signal and the electrically conductive fluid through one or more of the hollow needles, wherein electrically conductive properties of the fluid are suited to the tissue treatment; and removing the plurality of hollow needles from the dermal layer of skin.

In part, in yet another aspect, the disclosure relates to an apparatus for tissue treatment. The apparatus includes a housing comprising tissue contacting surface; an introducer disposed within the housing; a needle, at least a portion of the needle surrounded by the introducer; an electrical interface for coupling the needle and the introducer to a control system, the interface providing signals to: insert at least a portion of the needle into the tissue to create a tissue channel path; insert at least a portion of the introducer into the tissue channel path; retract the needle from the tissue channel path to separate a portion of the needle from a portion of the introducer disposed in the tissue channel path; and introduce fluid into the tissue channel path. In some embodiments, fluid is introduced into the tissue channel path by injection.

In part, in still yet another aspect, the disclosure relates to an apparatus for tissue treatment. The apparatus includes a housing comprising tissue contacting surface; one or more actuators; a plurality of elongate cylindrical tubes, wherein each tube defines a bore, wherein the plurality of tubes are disposed in the housing, wherein an opening of each tube is further defined by the tissue contacting surface; and an electrical interface. In various embodiments, each tube includes an introducer disposed within the bore of the tube; a solid needle, at least a portion of the needle surrounded by the introducer, wherein the needle and introducer are slidably disposed within the tube, the one or more actuators configured to extend each needle from each bore such that each needle extends and retracts relative to the tissue contacting surface. In many embodiments, the electrical interface is configured to electrically couple the needle and the one or more actuators to a control system, the interface providing signals to: insert at least a portion of the needle into the tissue to create a tissue channel path; insert at least a portion of the introducer into the tissue channel path; retract the needle from the tissue channel path to separate a portion of the needle from a portion of the introducer disposed in the tissue channel path; and introduce a fluid into the tissue channel path.

In various embodiments, the needle is electrically conductive and remains in contact with at least a portion of the fluid in the tissue channel path and RF energy is applied to the electrically conductive needle to treat the tissue adjacent the channel via electrical conduction through the fluid in the channel. In some embodiments, the needle is electrically conductive and after the introducer is actuated and prior to needle retraction, the RF energy is applied to the conductive needle to treat solely the tissue in contact with the needle tip. In some embodiments, the fluid temperature is controlled prior to introduction into the tissue channel path. In various embodiments, the fluid thermally modifies the tissue structure. In some embodiments, the thermal modification to tissue can be one or more of denaturation, apoptosis, stimulation, coagulation, necrosis, charring, and ablation.

In various embodiments, the fluid has electrical conductivity suited to the tissue treatment. In some embodiments, the target tissue is one of dermal tissue, subdermal fat, subdermal connective tissue, and mucosal tissue. In many embodiments, the apparatus further includes a plurality of needles each surrounded by an introducer to form a needle array within the housing. In some embodiments, the fluid is an electrically conductive fluid that modifies the tissue conductivity. In some embodiments, the electrically conductive fluid does not have analgesic properties. In various embodiments, the electrically conductive fluid causes biochemical changes to the tissue. In some embodiments, the fluid comprises one or more of a gas, a liquid, and a gel.

In some embodiments, controlling the temperature of the fluid determines whether the fluid comprises one or more of a gas, a liquid, and a gel. In some embodiments, the fluid modifies the tissue structure. In various embodiments, the needle is electrically conductive and radio frequency electromagnetic energy is delivered to needle in a bipolar mode. In some embodiments, the needle is electrically conductive and radio frequency electromagnetic energy is delivered in a monopolar mode. In some embodiments, one or more of the plurality of hollow needles has a cut angle that ranges from about 20° to about 70°, wherein one or more of the plurality of hollow needles defines an outlet and has a notch to regulate fluid ingress, the notch disposed above the outlet.

Although, the disclosure relates to different aspects and embodiments, it is understood that the different aspects and embodiments disclosed herein can be integrated, combined, or used together as a combination system, or in part, as separate components, devices, and systems, as appropriate. Thus, each embodiment disclosed herein can be incorporated in each of the aspects to varying degrees as appropriate for a given implementation. Further, the various systems, probes, applicators, needle arrays, controllers, components and parts of the foregoing can be used with any suitable tissue surface, cosmetic applications, and medical applications and other methods in conjunction with other devices and systems without limitation.

These and other features of the applicant's teachings are set forth herein.

### BRIEF DESCRIPTION OF THE FIGURES

Unless specified otherwise, the accompanying drawings illustrate aspects of the innovations described herein. Referring to the drawings, wherein like numerals refer to like parts throughout the several views and this specification, several embodiments of presently disclosed principles are illustrated by way of example, and not by way of limitation. The drawings are not intended to be to scale.

FIG. 1a is a plot of volume of solution, in nano liters, versus variable solution layer thickness, in μm, covering three needle tip designs with external diameter of 250 μm and cut angles of 60°, 45° and 30° according to an illustrative embodiment of the disclosure.

FIG. 1b is a side view of three exemplary hollow core needle tips having cut angles of 60°, 45° and 30°, respectively, according to an illustrative embodiment of the disclosure.

FIG. 1c is a perspective view of a deformable double sterile compartment or pouch according to an illustrative embodiment of the disclosure.

FIG. 1d is a perspective view of a treatment tip that includes additional needles for piercing and transporting a sterile fluid from a container such as shown in FIG. 1c according to an illustrative embodiment of the disclosure.

FIG. 1e is a perspective view of a disposable tip configured to interface and be received by a handpiece according to an illustrative embodiment of the disclosure.

FIG. 1f is a perspective view of a disposable tip interfaced with a handpiece with a compartment door according to an illustrative embodiment of the disclosure.

FIG. 1g is a perspective view of a disposable tip interfaced with a handpiece with a compartment door closed according to an illustrative embodiment of the disclosure.

FIG. 1h is a perspective view of a disposable tip interfaced with a handpiece before and during needle actuation according to an illustrative embodiment of the disclosure.

FIG. 2 is a schematic diagram showing different states of a microneedling device that corresponds to the general operation steps for using the microneedling device with hollow core microneedles designed to inject sterile salt solution for modification of the radio frequency (RF) power coupling from the microneedle tips to the surrounding tissue according to an illustrative embodiment of the disclosure.

FIG. 3a is a schematic diagram of a tip design with suitable for bipolar excitation of the hollow microneedles of the tip design with a pair of fluid reservoirs according to an illustrative embodiment of the disclosure.

FIG. 3b is a schematic diagram of a tip design with suitable for monopolar excitation of the hollow microneedles of the tip design with a pair of fluid reservoirs according to an illustrative embodiment of the disclosure.

FIG. 4a is an exploded perspective view of microfluidics tip with two fluid reservoirs according to an illustrative embodiment of the disclosure.

FIG. 4b is a perspective view of an assembled microfluidics tip with two fluid reservoirs according to an illustrative embodiment of the disclosure.

FIG. 5a is an exploded perspective view of microfluidics tip with two fluid reservoirs according to an illustrative embodiment of the disclosure.

FIG. 5b is a perspective view of an assembled microfluidics tip with two fluid reservoirs according to an illustrative embodiment of the disclosure.

FIG. 6a is a side view of an assembled microfluidics tip with a conformal coating and a notch for fluid delivery according to an illustrative embodiment of the disclosure.

FIG. 6b is a magnified side view of a microneedle that includes a conformal coating and a notch for fluid delivery according to an illustrative embodiment of the disclosure.

FIG. 6c is a magnified side view of a microfluidics tip that includes a needle that includes a notch for controlled fluid delivery and an electrical interface and control system according to an illustrative embodiment of the disclosure.

FIG. 7a is a schematic diagram showing different states of a microneedling device that correspond to the general operation steps for using the microneedling device with hollow core microneedles designed to be heated and inject a solution (such as an electrically or thermally conductive solution), such as a heated solution or steam, for modification of the RF power coupling from the microneedle tips to the surrounding tissue according to an illustrative embodiment of the disclosure.

FIG. 7b is a schematic diagram showing different states of a microneedling device that correspond to the general operation steps for using the microneedling device with hollow core microneedles designed to be heated and inject steam using a heated electrically or thermally conductive solution) for modification of the RF power coupling from the microneedle tips to the surrounding tissue according to an illustrative embodiment of the disclosure.

FIG. 8a is a schematic diagram showing different states of a microneedling device that corresponds to the general operation steps for using the microneedling device with hollow core microneedles designed to be heated and inject a sterile solution without use of an RF power coupling from the microneedle tips to the surrounding tissue according to an illustrative embodiment of the disclosure.

FIG. 8b is a schematic diagram showing different states of a microneedling device that corresponds to the general operation steps for using the microneedling device with hollow core microneedles designed to be heated and inject steam or otherwise expose target tissue regions to steam without use of an RF power coupling from the microneedle tips to the surrounding tissue according to an illustrative embodiment of the disclosure.

FIG. 9 is a perspective view of an exemplary assembly of a microfluidics tip with an array of hollow core microneedles for RF power delivery and heating chambers and heaters for fluid head up or vaporization according to an illustrative embodiment of the disclosure.

FIG. 10 is a bottom view of an exemplary microfluidics tip with two heating chambers and two check valve sockets according to an illustrative embodiment of the disclosure.

FIG. 11 is an exploded perspective view of an exemplary microfluidics tip with an array of hollow core microneedles for RF power delivery and heating chambers according to an illustrative embodiment of the disclosure.

FIG. 12 is an exploded perspective view of an exemplary microfluidics tip with an array of hollow core microneedles and heating chambers without RF power delivery according to an illustrative embodiment of the disclosure.

FIG. 13 is a schematic diagram showing different states of a microneedling device that correspond to the general operation steps for using the microneedling device with microneedles in the microneedle array comprising solid core conductive microneedles and hollow core non-conductive conduits (or introducers) according to an illustrative embodiment of the disclosure.

### DETAILED DESCRIPTION

Various embodiments of the disclosure relate to microneedles and arrays thereof that may be arranged or secured using various designs as part of a tip suitable for use in conjunction with applicator or handpiece. A given microneedle-based treatment device may deliver an electrically conductive fluid suitable to a tissue treatment. The microneedles may be in fluid and/or electrical communication with a treatment system. Individual microneedles are typically hollow and define a core or a fluid channel. An array of microneedles may be used to deliver RF energy in skin treatment. The treatment of skin may involve inserting an array of microneedles into a region of the skin to a pre-determined depth. Pulses of RF electrical power may be applied to one or more of the microneedles. The one or more microneedles function as electrodes in monopolar or bipolar modes to create regions of thermal damage and/or necrosis in the tissue surrounding the tips and/or the shafts of the needles.

Treatment with microneedles with electrically insulated shafts, often results in tissue thermal damage and coagulation in the upper region of the dermis that is in mechanical contact with the electrically insulated shafts. In addition, histological observations very rarely, if ever, show evidence of only localized tissue thermal damage or coagulation only at the skin depth targeted by the electrically non-insulated metal microneedles tips. The histological observations imply that the RF impedance between the metal shaft and the tissue surrounding the electrically insulating material on the metal shaft is lower than the RF impedance between the exposed metal tip and the surrounding tissue.

Applicant has discovered a modification in the microneedle array can be proposed that would allow for adjustable RF power coupling between the exposed metal tip and the surrounding tissue. Application of RF power in conjunction with various concentrations of NaCl solutions injected near the RF electrode such as a microneedle-based device as disclosed herein results in improved heating efficiency. In part, the disclosure relates to a microneedle array that includes hollow core needles with electrically insulated shaft's external surfaces can be used to deliver a very small amount, nL, of sterile NaCl solution at the tip of one or more (or every) microneedle followed by delivery of RF power.

In one aspect, the tip of the one or more microneedles can be positioned at a predetermined depth in the skin or in the sub-dermal fatty layer. A spacer, guard, or other device may be used to select the penetration depth and/or position of the microneedle array. The ability to select the penetration depth of the microneedle array supports or enables adjustable RF power coupling between the exposed metal tip and the surrounding dermal and/or fatty tissue. In various embodiments, the amount or the concentration of the NaCl solution can be varied to fine-tune the level of tissue thermal injury and thermal injury size or the level of induced tissue apoptosis, for example in the fatty tissue. In various embodiments, a portion of a needle has an insulator coating in a region at or above (or slightly below) tissue surface. The lower non-electrically insulated portion of a given needle is disposed in adipose tissue that also acts as an electrical insulator.

By using a hollow needle and releasing a conductive fluid while the needle is disposed in various tissue levels, the conductive fluid is configured to improve targeted heating or energy delivery at a particular tissue level and overcome some of the electrically insulative effects inherent in adipose and other subsurface tissues. Further, by optionally directing the RF or other energy more efficiently in regions below the surface, applicant has discovered systems, methods, and devices to reduce surface tissue heating/damage while more efficiently targeting non-surface tissue with specificity. In various embodiments, the delivery of subsurface conductive fluid is configured to increase energy delivery at a subsurface depth and reduce unwanted damage to tissue surface.

In another aspect, the disclosure relates to the advantages of NaCl solution or saline delivery or other conductive fluids with hollow core microneedle arrays and the heating of such fluids via being in thermal communication with a heat source or through the application of RF energy or other mechanisms and devices. In various embodiments, analgesic or anesthetic can be added to the NaCl or saline solution for patient pain mitigation in response to delivery via the hollow core microneedles. Pain mitigation may be considered as a multiple step process. In the first pass over a treatment area the small amount of analgesic or anesthetic injected through the one or more hollow core microneedles into a treatment area and will have limited effect in the immediate vicinity of the microneedle. In subsequent pass(es) over the treatment area the analgesic or anesthetic will have time for diffusion in the surrounding tissue and provide improved pain mitigation.

In various embodiments, application of hollow core microneedle arrays use microneedles that have an external diameter that ranges from between about 200 μm and about 250 μm. Table 1 provides, from Sigma Aldrich, shows examples syringe needle suitable for use with embodiments of the disclosure that have dimensions for gauges from 31 to 33.

| Needle | Nominal | Nominal | Nominal |
|---|---|---|---|
| Gauge | O.D., mm | I.D., mm | Wall., mm |
| 31 | 0.254 | 0.114 | 0.064 |
| 32 | 0.229 | 0.089 | 0.051 |
| 33 | 0.203 | 0.089 | 0.051 |

The mechanical strength of the hollow core microneedle array is selected such that it is suitable for about 1000 skin insertions in some embodiments. In various embodiments, the mechanical strength of the needle array is selected to meet or exceed various performance targets relating to amounts of skin insertions.

Current RF microneedling technology uses a disposable sterile microneedling array tip attached to a non-sterile handpiece delivering RF power and mechanical force for insertion of the microneedling array in the skin. It would be preferable to have the sterile NaCl or saline solution fully contained in the sterile tip and using microfluidics to deliver small amounts, nL, of solution during every insertion for a maximum of 1000 or more insertions. In various embodiments, one or more components of the microneedle-based device such as the needle array are disposable. Various electrically conductive fluids may be used such as NaCl or saline solution. The fluids may be stored in the sterile tip may be formulated as a gel for manufacturing and storage. The gel may be advanced through the hollow core needles using some small fraction of the RF or mechanical power delivered to the tip. A pump or other system for pressurizing and delivering the fluid through the needles may be used in various embodiments. The movement of the gel or liquid solution may be designed on the basis of a microfluidics system. Other examples of solutions that can modify the needle-to-tissue power coupling include variable concentration KCl, as well as other inorganic salts, dissolved in water.

A few non-limiting examples of the underlying concepts and capabilities of microfluidics systems are given in Beebe, David J., Glennys A. Mensing, and Glenn M. Walker. “Physics and applications of microfluidics in biology.” Annual review of biomedical engineering 4.1 (2002): 261-286; Squires, Todd M., and Stephen R. Quake. “Microfluidics: Fluid physics at the nanoliter scale.” Reviews of modern physics 77.3 (2005): 977-1026; and Karbalaei, Alireza, Ranganathan Kumar, and Hyoung Jin Cho. “Thermocapillarity in microfluidics-A review.” Micromachines 7.1 (2016): 13, the disclosure of which are incorporated by reference in their entirety.

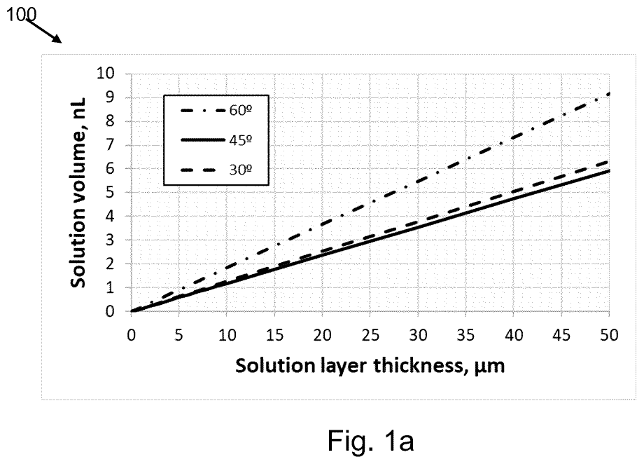

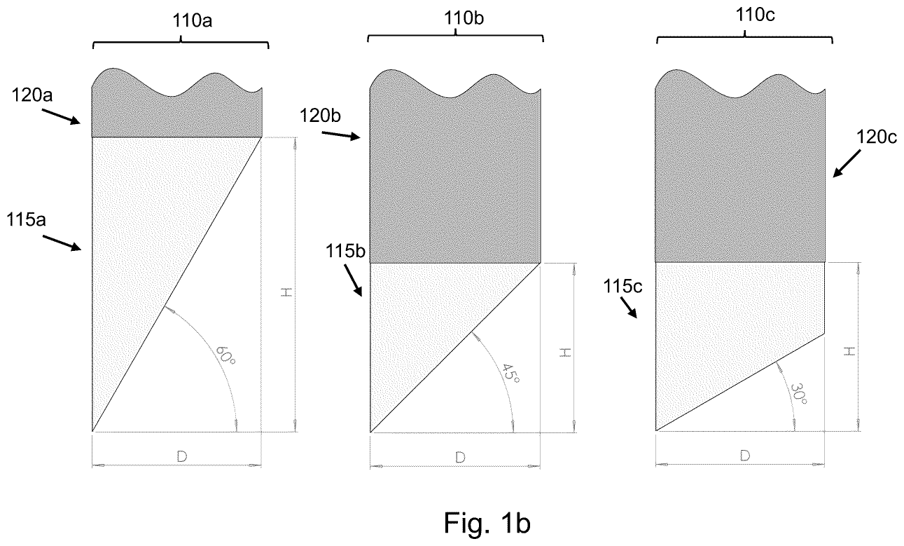

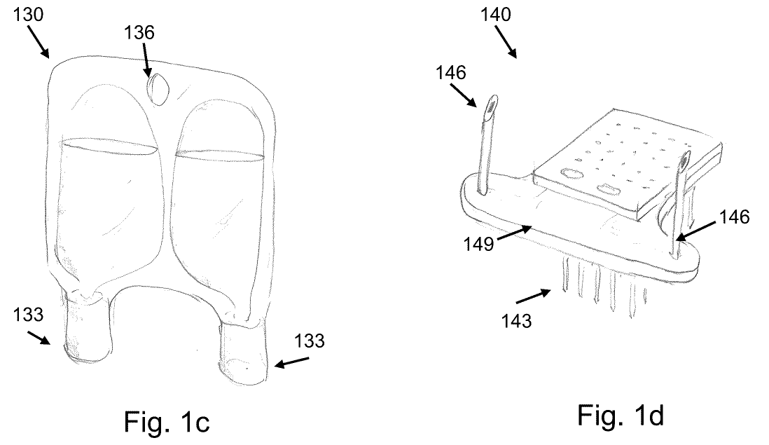

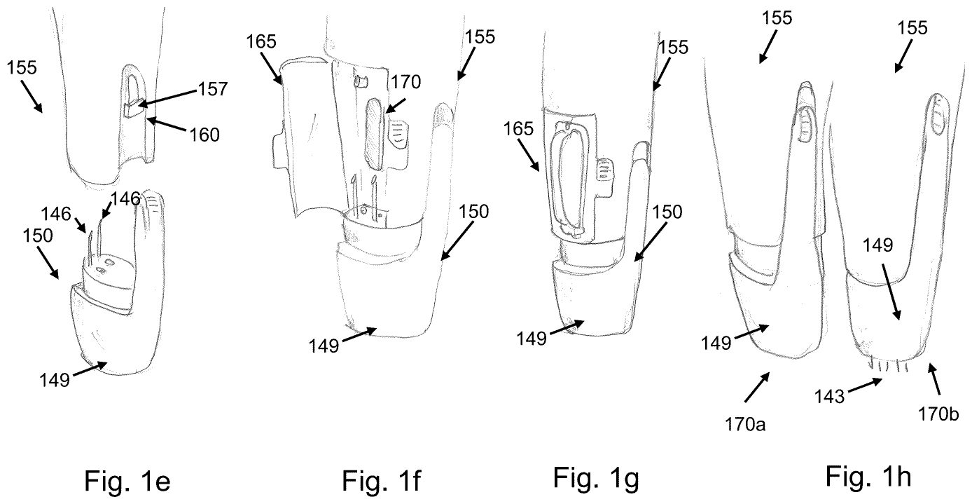

FIG. 1a shows a graph 100 of the calculated approximate volume of solution with variable solution layer thickness (in μm) that will cover a needle tip with an external diameter of D=250 μm and cut angles of 60°, 45° and 30°. FIG. 1b shows three non-limiting examples of needle tips 110a, 110b, 110c cut with angles of 60°, 45° and 30°. FIG. 1b shows the non-insulated, exposed, region 115 of the needle tips in contact with the variable solution layers are with an exposed needle tip height H and diameter D. The needle tips 110 may include an electrically insulated region 120 as shown in brown. For cut angles below 45° the exposed needle tip height H is set to be approximately equal to the needle diameter D (i.e., D≈H).

In some embodiments, sterile saline can be pre-packaged in sterile containers with deformable or perforable walls. The sterile container includes a delivery tip or tips designed to provide leak proof sterile connection to the sterile treatment tip when the tip is attached to the handpiece. Immediately before use, the sterile container with deformable walls filled with sterile saline can be inserted in the non-sterile handpiece so that the delivery tip does not get contaminated by the handpiece walls or other surfaces. Then the sterile treatment tip is connected to the handpiece forming a leak proof sterile connection with the sterile tip of the saline container. The saline container or other containers of treatment fluid may be various shapes such as tubes, cylinders, spheres or other shapes suitable for containing sterile fluid for delivery by the hollow needle array. The containers of treatment fluid can include electrical and/or thermally conductive materials and may have other additives such as analgesics, medicaments, and other chemicals that support the nature of the treatment or cosmetic procedure being performed on a given subject.

FIG. 1c shows a sterile container with 130 deformable walls. The sterile container 130 is capable of storing sterile saline solution. The sterile container 130 may include tips 133 which interface with the sterile treatment tip to provide a leak proof sterile connection. The deformable container 130 may also include an alignment hole 136 which is used to align the deformable container 130 with the handpiece. The container may include various alignment features such as a hole or others. The container can be implemented using various fluid containing vessels. The container may be a pouch with two fluid output ports and is configured as a small double IV bag sized to fit in the handpiece. The container 130 can be filled with any suitable sterile fluid such as, for example, electrically conductive and thermally conductive fluids suited to tissue treatment(s) as disclosed herein.

FIG. 1d is a perspective view of a treatment tip that includes additional needles for piercing and transporting a sterile fluid from a container such as shown in FIG. 1c according to an illustrative embodiment of the disclosure. In particular, FIG. 1d shows a treatment tip 140 which includes hollow microneedles 143, and sterile saline solution transport needles/tubes 146. The treatment tip apparatus 140 is configured to interface directly with a sterile container, for example, the sterile container 130 shown in FIG. 1c. The hollow microneedles 143 are disposed in or are otherwise attached to or supported by a base 149. Needles 143 and 146 may extend from or interpenetrate the base 149 to varying depths. The base 149 may include one or more electrical contacts and electrical leads in communication with all or a subset of the needles 143. The base can be various shapes, for example, the base may be square, rectangular, round, curved, or combinations of various shapes, edges, and curved or straight lines without limitation.

FIG. 1e shows a microneedle-based tip 150 configured to interface and attach or couple with a handpiece 155. The microneedle-based tip 150 includes a base 149 through which microneedles 143 may slidably move relative thereto. The handpiece 155 includes slide mechanism 160 that couples to the disposable tip to activate the needles into a patient's skin. The microneedle-based tip 150 may be disposable.

FIG. 1f shows a disposable tip 150 interfaced with or otherwise attached to a handpiece 155. The tip includes the base 149 or the tip and base may be the same structure in some embodiments. The handpiece 155 includes a compartment door 165 such as door in a side panel or surface of the handpiece which opens to exposes a compartment capable of housing a sterile container. As shown in the interior of the handpiece 155, a plate 170 or other force delivery device is disposed within the handpiece. The plate is configured to advance or otherwise translate to squeeze the sterile container against the compartment door 165 or another static element adjacent the deformable container. In some embodiments, the plate 170 or other force delivery device is synchronized with the RF pulses or another parameter associated with treatment such that the sterile container is squeezed such that fluid is delivered with each treatment pulse or based on another time period or frequency associated with tissue treatment using microneedle tip. FIG. 1g depicts the disposable tip 150 and handpiece 155 when the compartment door 165 is closed.

FIG. 1h shows the disposable tip and handpiece before 170a and after 170b needle actuation. Needles 143 extend and retract relative to an array of holes or aperture in the base 149 150. The disposable tip and sterile container stay positively connected during needle actuation in some embodiments. In various embodiments, a plastic housing of the needle array travels, slides, or otherwise moves to push the needles into the skin.

In some embodiments, during treatment the handpiece exerts mechanical force and deforms the wall of the saline container in order to deliver sterile saline into the treatment tip. The movement of the sterile saline through the treatment tip and the delivery of saline through the hollow microneedles is enabled by a microfluidics system embedded in the treatment tip. In various embodiments, reference to saline also encompasses other suitable treatments, electrically conductive, and/or thermally conducive fluids, gels, gases, vapors and other fluids disclosed herein.

In various embodiments, small quantities of saline or gel at elevated temperature, between 30° C. and 80° C., can be delivered through the array of hollow core microneedles. The combined effects of the small quantities of saline at elevated temperature and the RF power delivered through the microneedles may lead to the formation of regions of thermal damage and/or necrosis in the tissue surrounding the tips and/or the shafts of the hollow core microneedles. In some embodiments, without RF power, the effect of the small quantities of saline at elevated temperature may be sufficient to lead to the formations of regions of thermal damage and/or necrosis in the tissue surrounding the tips and/or the shafts of the hollow core microneedles. In other embodiments the effect of the small quantities of saline at elevated temperature combined with a relatively lower power RF generator incorporated into a lower cost device that delivers similar biological effects as a higher power, more expensive, RF generator without delivery of saline at elevated temperature. In some embodiments, analgesics or other chemicals are also delivered to the tissue or the tissue surface using the needles or via another fluid delivery mechanism. Various types of cooling may also be applied in conjunction with the microneedling process. In some embodiments, the volumes of delivered fluid and solution layer thickness may be selected in accordance with FIG. 1a.

In various embodiments, the non-sterile handpiece contains a water reservoir, and it is designed to generate sterile steam that can be delivered to the sterile treatment tip and through the array of hollow core microneedles. A microfluidics system in the treatment tip delivers small quantities of steam to the tips of the hollow core microneedles. The delivered small quantities of steam and its temperature are selected so that it is sufficient to lead to the formations of regions of thermal damage and/or necrosis in the tissue surrounding the tips and/or the shafts of the hollow core microneedles. In some embodiments, RF power is delivered to the array of hollow core microneedles in conjunction or in addition to the steam delivery.

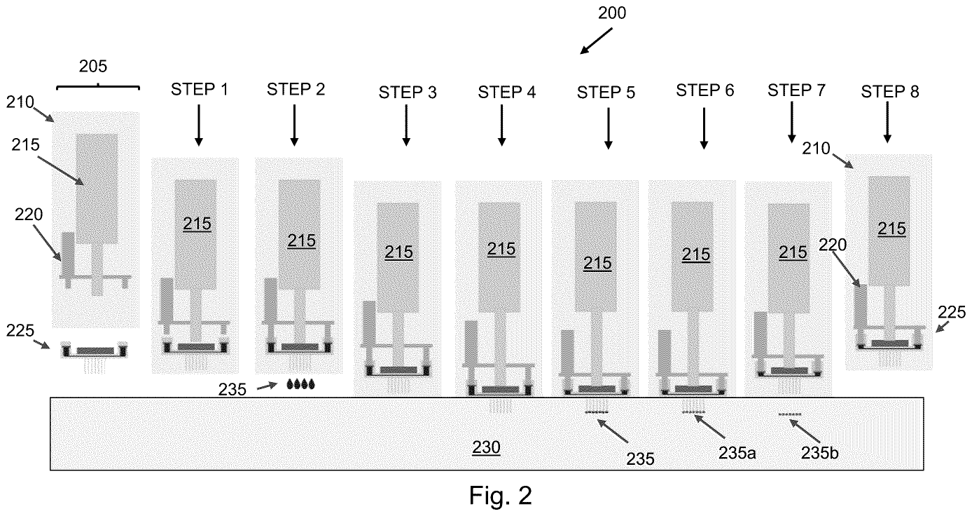

Refer now to FIG. 2, which depicts a non-limiting example of a method 200 of various general operation steps for a microneedling assembly device with hollow core microneedles and a device 205 for performing the steps of the method. The various components of an exemplary microneedling device 205 are shown in FIG. 2 and followed by the various steps that may be performed relative to the device 205. The device 205 may include one or more of a non-sterile handpiece 210, a needle array actuator 215 and a piezo micro-actuator 220, a sterile microneedling tip 225 pre-loaded with a sterile saline solution. In various embodiments, the sterile microneedling tip 225 is disposable.

In Step 1 a non-sterile handpiece 210 including, a needle array actuator 215 and a piezo micro-actuator 220 are combined with a sterile microneedling tip 225 pre-loaded with a sterile saline solution. In various embodiments, the sterile microneedling tip 225 is disposable. In Step 2 the assembled device is “primed” before use on a region of patient skin 230. In Step 2, small amounts of saline 235 are pushed out of one or more microneedles to verify correct handpiece-and-tip assembly and operation of the piezo micro-actuator 220 and the microfluidics system in the sterile microneedling tip 225. Step 2 is optional and may not be performed in various embodiments. In Step 3, the handpiece-and-tip assembly is positioned on the patient's skin 230 in the treatment area. In Step 4 the needle array actuator 215 pushes the microneedling tip 225 into the patient skin 230 down to a pre-set depth or deeper into the patient subdermal fatty layer. In Step 5, pre-determined quantities of saline are injected by the one or more hollow core microneedles.

In various embodiments, saline is injected while monitoring the impedance of the microneedling tip 225 in the tissue and injection is stopped when a pre-determined impedance level is reached. Optionally, the saline injection may start during Step 4 while the microneedling tip 225 is moving though the patient skin and some of the saline is injected along the microneedling tip 225 path through the patient skin 230. The injected saline 235 is shown in Step 5 after the solution has been injected. In Step 6, RF power is turned on for a pre-determined on-time and injected saline 235 conducts RF power as shown by residual saline 235b which may be heated as a result of the application of RF to the tissue. In various embodiments, the RF power level and on-time is variable and determined by a control system based on real-time measurements of impedance and/or tissue temperature during RF power delivery. In Step 7, the microneedling tip is retracted from the skin with the delivered saline 235b remaining or being absorbed by the tissue. In Step 8, the handpiece-and-tip assembly is lifted from the patient tissue 230 and is prepared to be positioned on the next treatment spot on the patient tissue (such as the patient skin).

In various embodiments, the RF power delivery to the microneedle array can be designed to be in bipolar or monopolar mode. In the bipolar mode of RF power delivery, a first group of the microneedles in the microneedle array are connected to one pole of the RF generator and the rest of the microneedles, the second group, are connected to the other pole of the RF generator. If the first and the second groups of microneedles are connected to a common reservoir of saline, the conductivity of the saline in the microfluidics tip may be sufficient to create a low impedance, or short, connection between the two poles of the RF generator through the common reservoir. In the presence of such short connection most or all of the RF power may be delivered to the saline reservoir and/or the microfluidics tip and not to the patient treatment area. Such short connection can be avoided, for example by having a tip design with two separate reservoirs in the sterile tip.

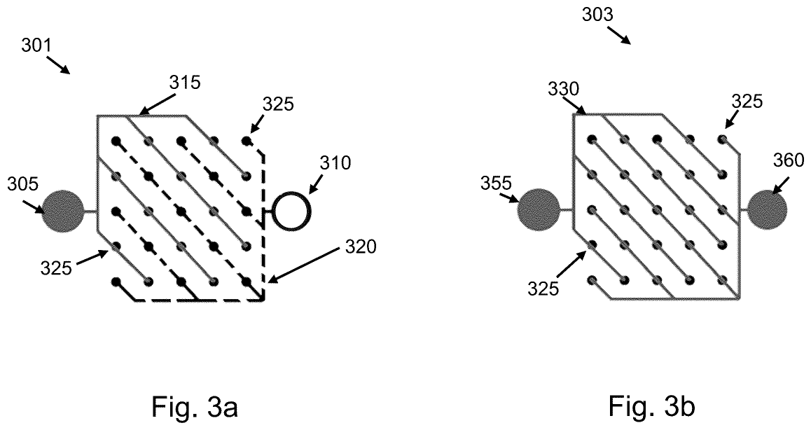

FIGS. 3a and 3b are schematic diagrams of a tip design with suitable for monopolar excitation of the hollow microneedles of the tip design with a fluid reservoir for (a) bipolar and (b) monopolar operation according to an illustrative embodiment of the disclosure. FIG. 3a shows the bipolar excitation of needles with two independent injection paths. FIG. 3b shows monopolar excitation of needles with two independent injection paths that can be connected to form a continuous flow path.

Turning to FIG. 3a, an example of a tip design 301 is depicted that includes two separate reservoirs 305, 310 shown as a large solid black 305 circle and a circle with a white interior 310. In some embodiments, reservoirs 305 and 310 may include the same or different fluids or other materials. The microfluidics paths 315, 320 shown as solid black lines 310 and doted lines 320 are arranged so that each of the microneedles, shown as black dots 325, has as nearest-neighbor-microneedles of opposite polarity. Microfluidics paths 315, 320 are interleaved and arranged in a symmetrical pattern. In some embodiments, microfluidics paths 315 (solid lines), 320 (dotted lines) are arranged in an alternating parallel pattern. In some embodiments, microfluidics paths 315, 320 are each defined by a plurality of interleaved diagonally arranged fluid channel segments.

FIG. 3b shows an example of a tip design 303 with two reservoirs 355, 360, shown as large solid black circles, for monopolar mode of operation. In the case of monopolar mode of RF power delivery, a return electrode, or NEM pad, is positioned on the patient skin and all the microneedles 325 in the treatment tip have the same polarity. In that case a single saline reservoir is sufficient for monopolar mode of RF power delivery. One benefit illustrated by the design of FIG. 3b is that one tip can be used both in monopolar and bipolar mode of operation and therefore simplifying tip inventory management. As shown in FIG. 3b, one continuous fluid flow path/injection path 330 can be used that can be formed from two separate injection paths that can be connected to form path 330. The two separate injection paths may correspond to paths 315, 320 shown in FIG. 3a, but in FIG. 3b they are connectable or connected. In FIG. 3b, fluid injection path 320 is shown by dotted lines for visibility. Fluid injection paths 315, 320 are a continuous fluid path or flow path in various embodiments. In some embodiments, reservoirs 355 and 360 may include the same or different fluids or other materials.

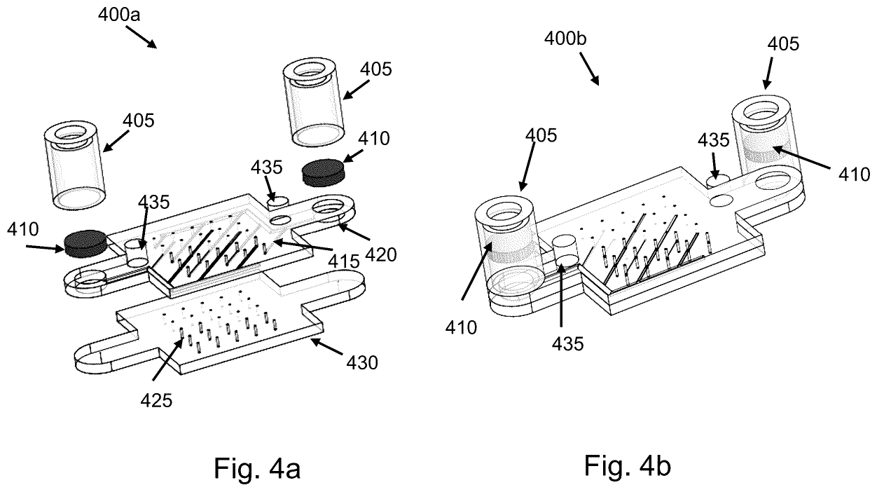

FIGS. 4a and 4b depict example designs for a microfluidics assembly 400a, 400b with two reservoirs. FIG. 4a, depicts an exploded view of the microfluidics assembly 400a, while FIG. 4b depicts an assembled microfluidics assembly 400b. Each of the cylindrical reservoirs 405 has a plunger 410, shown as a disk or cylinder The microfluidics channels 415 are part of the upper component 420 of the assembly. Microneedle array guide holes 425 are made in both the upper component 420 and the lower component 430 of the assembly. Various alignment elements 435 such as nubs or posts may be included in the microfluidics assembly to hold and/or align the PC board in place. FIG. 4b shows the assembled microfluidics components. In various embodiments, the components and the microfluidics assembly can be formed from various polymer-based materials.

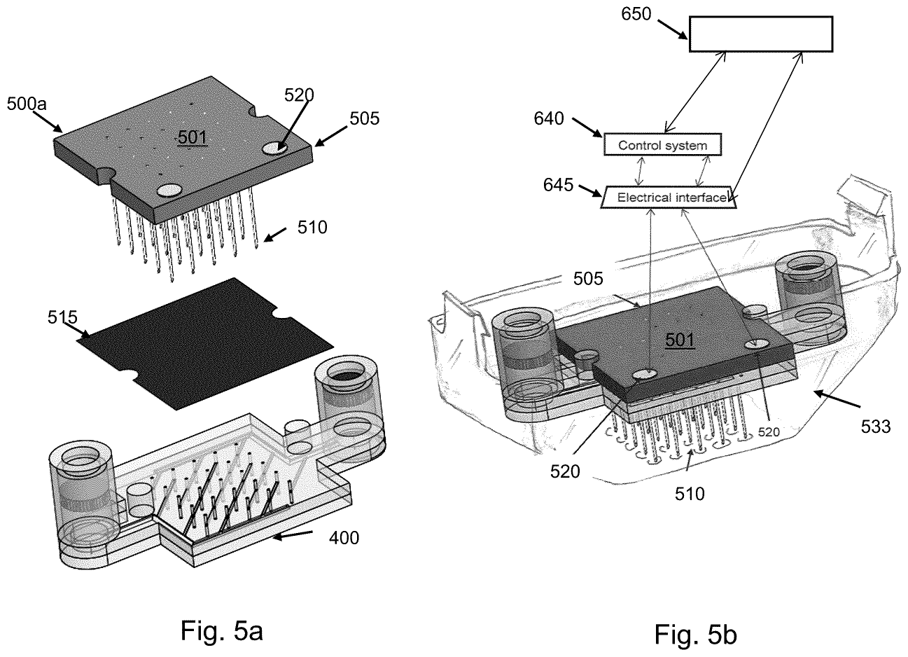

FIGS. 5a and 5b depict example designs of the microfluidics tip assembly 500a, 500b in which the microneedling tips have been included and disposed in a base 501 of array 505. In some embodiments, the base is a printed circuit board or includes a printed circuit board. A given circuit board may include electronic traces and electronic components to receive RF power and/or control signals from a power supply and control system, respectively. FIG. 5a depicts an exploded view of the microfluidics tip assembly 500a and FIG. 5b depicts an assembled microfluidics tip assembly 500b. In FIG. 5b a guard 533 is shown configured to support, enclose, or receive one or more components. In various embodiments, the guard 533 includes a housing and has a group of holes defined therein such that the needles may extend from and retract relative to such holes and the tissue contacting surface of the guide 533. An array 505 of hollow core microneedles 510 includes suitable electrical connections for bipolar or monopolar operation. Optionally, the shaft of each of the hollow core microneedles 510 is coated with an electrical isolator layer, for example a layer of parylene coating. The shaft electrical isolator layer leaves a non-isolated about 10 to about 500 μm long region at the tip of each hollow core microneedles 510. In various embodiments, the array 505 includes a base 501 or other support that contacts or supports the needles of the array 505. The array 505 is inserted in the assembled microfluidics assembly 400 depicted in FIGS. 4a and 4b through a top gasket 515 designed to prevent fluid leakage. Electrical contacts 520 that are in electrical communication with the needles of the needle array are configured to receive RF signals, control signals, and other electrical signals from an electrical interface and/or a control system.

FIG. 5b shows the assembled microneedle array and microfluidics components and also shows an electrical interface 645 and a control system 640. In one embodiment, the control system includes a user interface that seeks and receives user input, necessary control electronics, and indicators (e.g., a display revealing the system status such as power coupling to the targeted tissue). In some embodiments, the electrical interface is configured to electrically couple the needles with the power supply 650 (e.g., the RF power supply). The electrical interface may include actuators for moving fluid through the device, for example, valves, motors, piezotransducers, and heaters.

The guard 533 depicted in FIG. 5b may be part of the tip assembly that it encloses, and the needles can be extended or retracted relative to the holes in the housing of the guard. The guard may include a housing having various shapes and configurations. The guard is removably coupled to a handpiece in some embodiments. The guard protects the complex electro-mechanical and fluid delivery mechanisms disposed therein from impact while also protecting the patient from contacting such mechanisms directly. Additionally, the guard supports and maintains the alignment of the various translatable components of the microneedle assembly or tip disposed therein. In various embodiments, the guard is constructed, such as by molding, using a plastic or other patient-safe polymer material.

In various embodiments, the interior of the guard may include posts or nubs suitable for further enhancing alignment of the components disposed therein and with the handpiece to which the guard may removably connect. In various embodiments, one more components disposed in the handpiece are disposable and the guard is configured to release from and attach to the handpiece to facilitate the replacement of any of the disposable components or to otherwise adjust any adjustable components or switches within the handpiece. In some embodiments, the switches may control which microfluidic pathways are used for a given treatment session. In some embodiments, the guard is directly attached to the handpiece or unitary with the handpiece, but the surface of the guard and/or handpiece includes a removable element such as a slidable and removable sidewall or a hinged wall or other access mechanism that may open to support access to the interior of the micro-needling device to remove or add components such as disposable components.

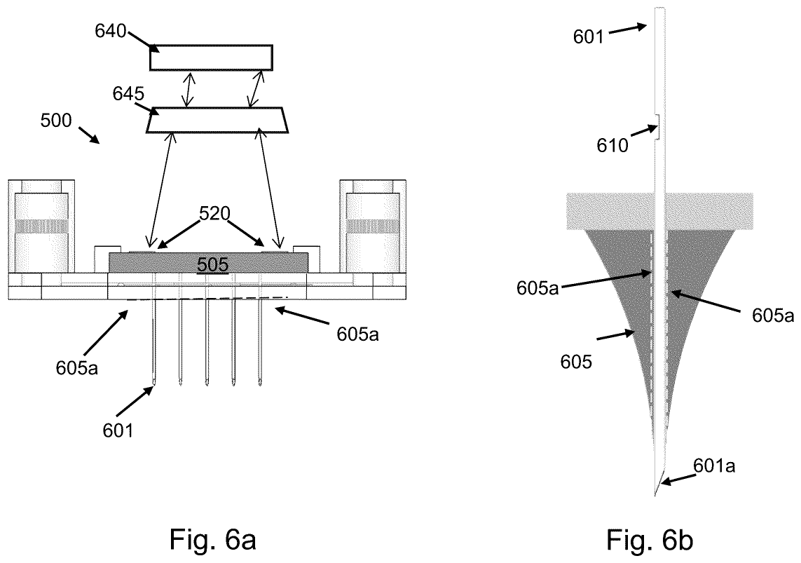

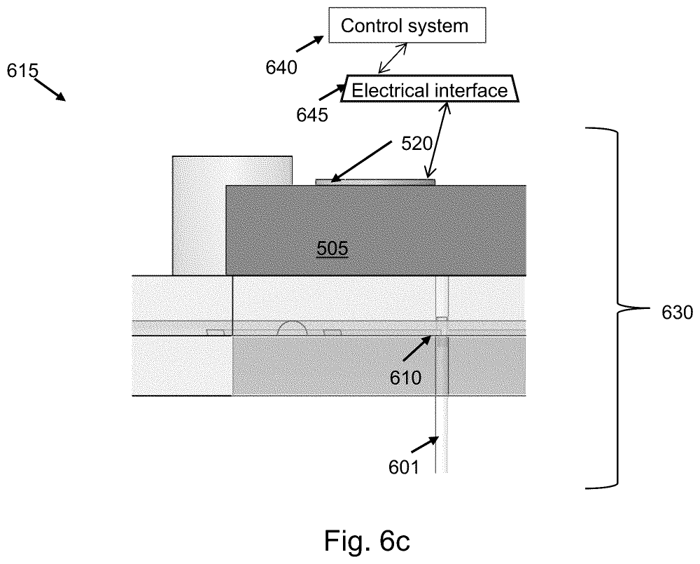

FIG. 6a shows an exemplary coating or layer to prevent fluid leakage through the lower component of the microfluidics assembly that includes various needles 601 that each define a bore or fluid channel. A given needle 601 may include an outlet 601a that operates to deliver fluid flowing through the bore or hollow channel of the needle. For example, a conformal coating 605, 605a may be applied to the tip assembly serving both to prevent fluid leakage and providing electrical isolation on the microneedle shafts. For example, the conformal coating material may include parylene. Exemplary conformal coating 605a is shown by a dotted line in FIG. 6a and FIG. 6b. In some embodiments, the conformal coating such as shown by exemplary conformal coating layer 605a, the layer 605a is closely deposited on the outer surface of needle 601. In other embodiments, the conformal coating is thicker and may have a wider base that tapers along the needle as shown by the schematic representation of conformal coating 605. The conformal coating that is applied may be invisible upon physical inspection or thick enough to be seen in some embodiments. In some embodiments, ultrasound welding of microneedles to the plastic lower microfluidics component can be used to prevent fluid leakage. In various embodiments, the conformal coating has a thickness that ranges from about 200 μm to about 1 mm. A control system 640 is in communication with an electrical interface 645. The thickness of the conformal coating can be substantially the same such as shown with layer 605a or have a taper or other variable thickness when deposited or formed on a given needle. The electrical interface 645 and/or the control system are in electrical communication with one or more electrical contacts of the tip.

FIGS. 6b and 6c show details of the microfluidics assembly in which a notch 610 is cut or is in or defined by the wall of one or more hollow core microneedles 601. The notch 610 defines a fluid transport path from the bore of the microneedle to a fluid source such as a reservoir. The notch may be configured for fluid delivery from the microfluidics channels to the microneedle cores/bores. In various embodiments, the notch supports efficient alignment of a fluid path with the needle while also allowing needle to remain aligned for any electrical connections with the electrical interface. The system 615 shown in FIG. 6C includes a partial schematic view of the microfluidics assembly 630 that includes electrical contacts 520. A control system 640 is in electrical or wireless communication with an electrical interface 645. The electrical interface 645 is in electrical communication with electrical contacts 520. In various embodiments, movement of the needle assembly allows the notch 610 of each needle (or a subset thereof) to engage with a fluid path that connects to a fluid reservoir or source of steam such that fluid or steam may flow into the notch and be delivered to tissue from the opening of each microneedle.

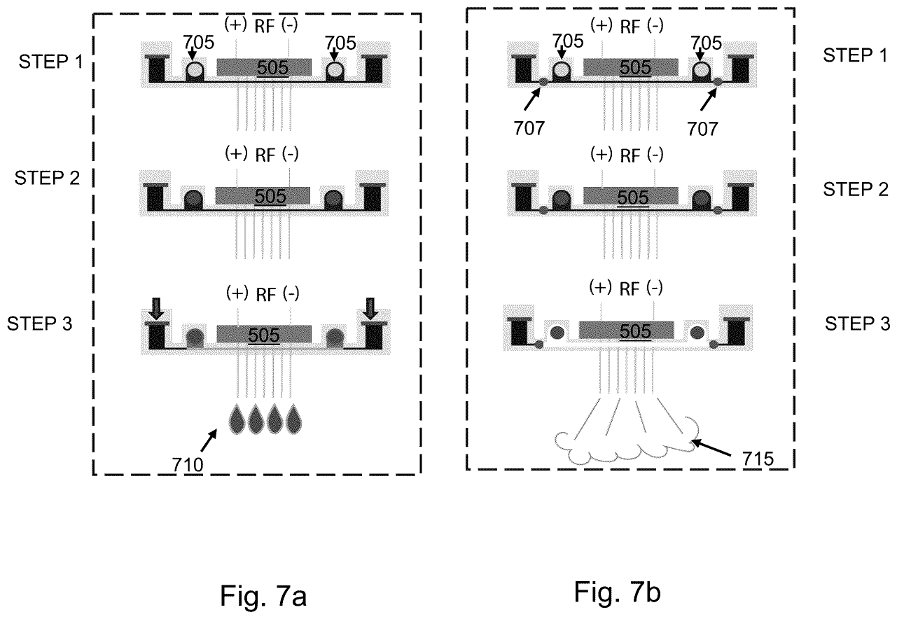

A non-limiting example of the operation of an RF microneedling array with heated treatment solutions, heated conductive solutions, or other solutions such as heated saline is illustrated in FIG. 7a. A non-limiting example of the operation of an RF microneedling array in which steam or a vaporized solution is delivered is illustrated in FIG. 7b. The main steps shown in FIGS. 7a and 7b are substantially the same with the main difference being the delivery of a solution versus steam or a vaporized solution. Step 1 depicts the RF microneedling array that includes compact heaters 705 in which the compact heaters 705 are turned off. In Step 2, which occurs after the needle priming step discussed with respect to FIG. 2, one or more compact heaters 705 are turned on for sufficient time to heat up a given solution, such as a saline solution, to a predetermined temperature between 30° C. and 80° C., FIG. 7a; or convert the fluid to steam, FIG. 7b. In Step 3, the heated saline 710 is pushed through the hollow core microneedles by the microfluidics system, FIG. 7a; or the steam 715 expansion pushes a predetermined amount of steam through the hollow core microneedles, FIG. 7b. In various embodiments, delivery of the steam 715 may be controlled by one or more valves 707 such as inline valves. After the delivery of the heated saline, or steam, the RF power is delivered for a predetermined on-time or based on tissue temperature and/or impedance real-time feedback. In some embodiments, in lieu of steam, a mist or other vaporized solutions such as is derived through ultrasonic vaporization may also be used in lieu of heated steam.

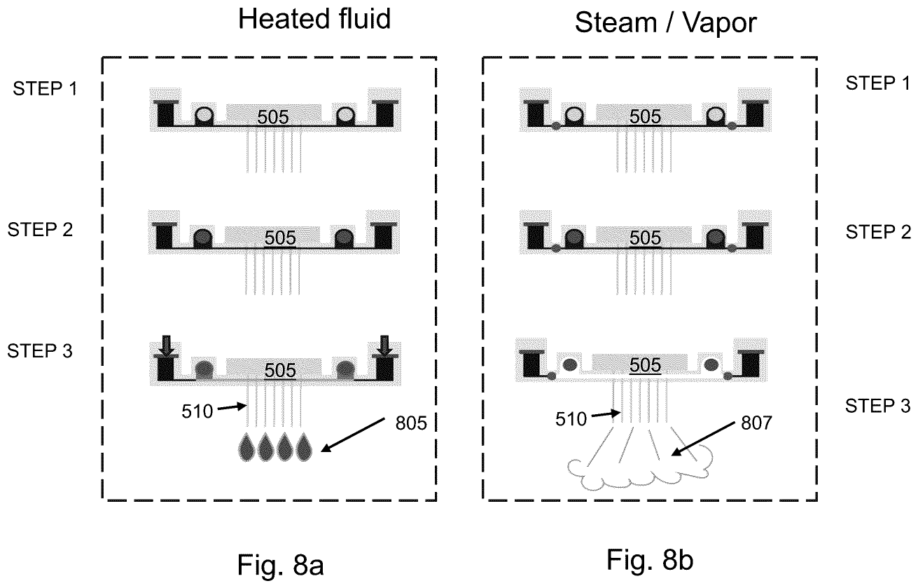

Refer now to FIGS. 8a and 8b, which depicts operation of a microneedling array with heated saline (or other heated fluids) or steam without delivery of RF power, respectively. In this embodiment, the heating and fluid delivery steps follow the embodiment discussed with regard to FIGS. 7a and 7b. However, in this embodiment, in contrast with RF-based embodiments, the desired tissue thermal effects, or necrosis, are caused only by the tissue interaction with the heated saline or steam without contribution from RF power delivery.

FIGS. 8a and 8b shows two similar exemplary methods that includes various operational steps for a microneedling device with hollow core microneedles designed to inject a different material. As shown in FIG. 8a, a heated up sterile salt solution 805 is injected. Similarly, as shown in FIG. 8b steam 807 is injected. The injection of a heated solution or steam lead to formation of regions of thermal damage and/or necrosis in the tissue surrounding the tips and/or the shafts of the microneedles without RF power delivery according to an illustrative embodiment of the disclosure. In various embodiments, disclosed herein a cooling plate or other cooling mechanisms, methods and devices may be used to pre-cool the skin or cool the skin during or after microneedling prior to injected a heat solution, such as a saline or other solution, or steam as disclosed herein.

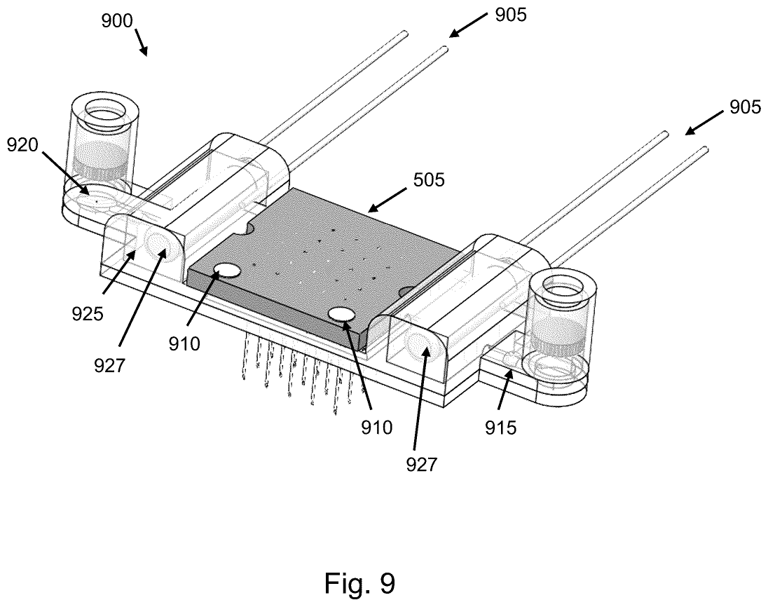

Refer now to FIG. 9 which depicts an example design of a microfluidics tip 900 incorporating RF power delivery and fluid heaters. The microfluidics assembly 900 includes heater power source 905, and RF connections 910. In various embodiments, one or more check valves 915 separate the fluid or gel reservoir(s) 920 and the heater chambers(s) 925. These valves are included to prevent backflow or otherwise support or control fluid delivery. The inclusion of the check valves advantageously supports fluid control and mitigates the risk of contamination and other unwanted effects. The purpose of the check valve 915 is to allow fluid delivery from the reservoir 920 to the heating chamber 925 and prevent back flow from the chamber 925 back to the reservoir 920. The fluid heaters 927 can be of various suitable designs such as the elongate heaters shown and are typically disposed in heating chamber 925 or in fluid communication therewith.

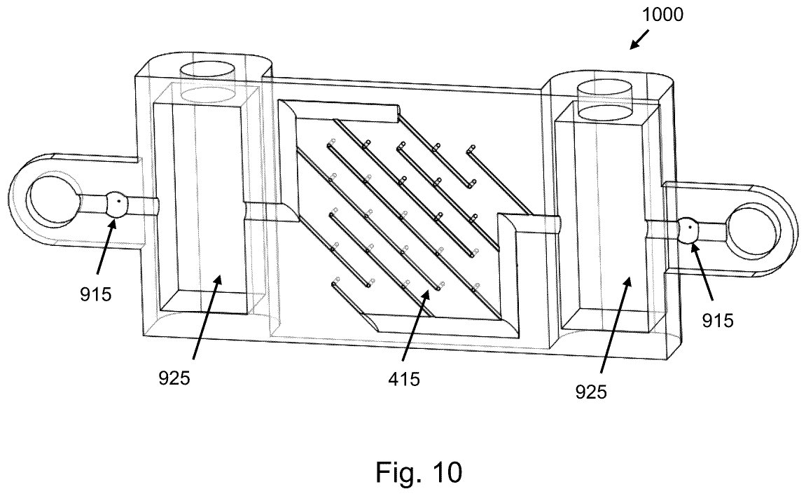

Refer now to FIG. 10 which depicts the microfluidics assembly 1000 with the heating chambers 925 and check valve sockets for the check valves 915 in detail. The design outlined in FIG. 10 is applicable to microfluidics microneedling assemblies that deliver heated, or vaporized, fluid or gel with or without RF power delivery.

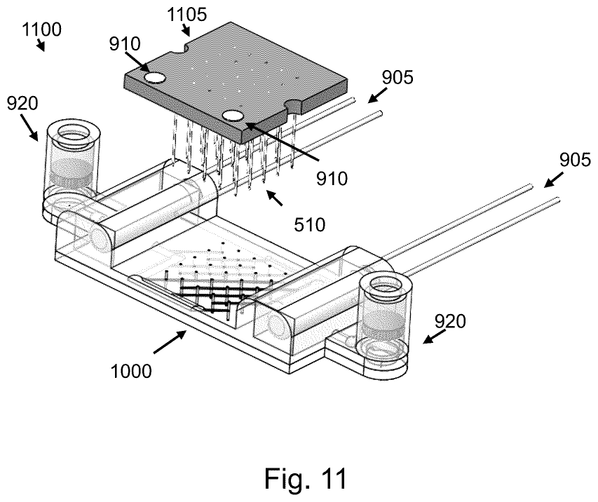

Refer now to FIG. 11 which depicts an exploded view of a microfluidics microneedling assembly 1100 with heated up, or vaporized, fluid or gel with a PCB 1105 configured for RF power delivery. In some embodiments, a base of the that is attached to or supports a plurality of hollow needles includes the PCB 1105. The set of needles 510 is sized to be received by the holes of microfluidics assembly 1000 and move up and down relative to a surface of the assembly 1000.

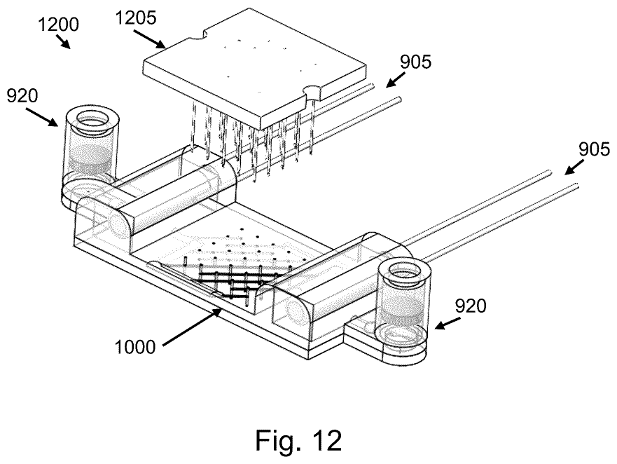

Refer now to FIG. 12 shows an exploded view of a microfluidics microneedling assembly 120 suitable for use with heated, or vaporized, fluid or gel or other material with a PCB 1205 that is not configured for RF power delivery or for which the ability to deliver RF power can be selectively turned on or off.

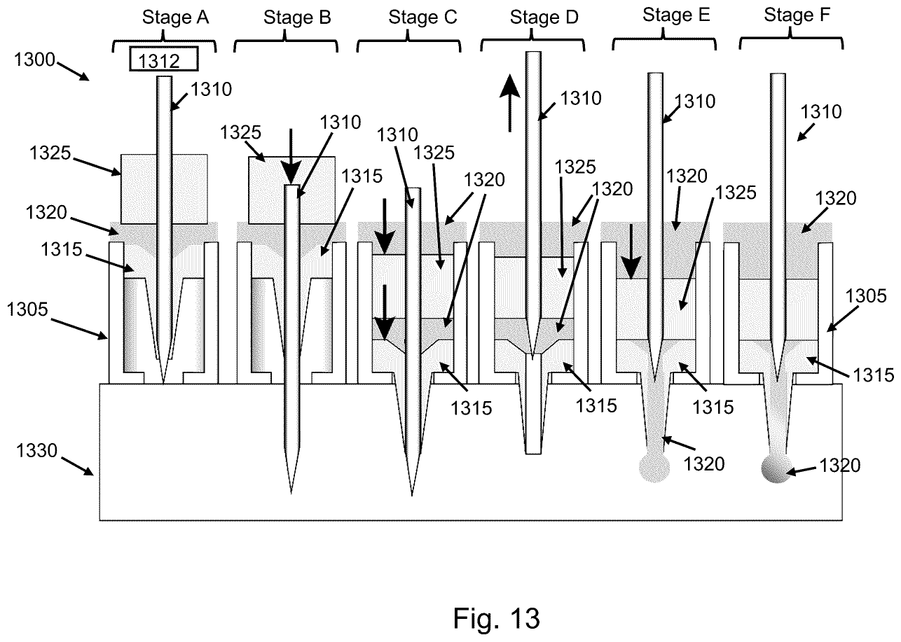

Refer now to FIG. 13 which depicts an exemplary hollow core microneedle during operation as it progresses through various operational stages 1300 in which a fluid is delivered to tissue such as within or below a skin tissue layer. Although the various stages of one exemplary hollow core microneedle during operation are shown, the stages generally apply to some or all of the needles present in a given needle array or assembly as disclosed herein. Various directional up and down arrows are also shown in the various stages to indicate directions of travel, motion, or relative motion of device components. The various stages are shown in a generalized partial side view in FIG. 13.

In stage A of FIG. 13, the arrangement of various components of a microneedle assembly is shown. A disposable sterile housing 1305 is shown that defines an inner cavity or bore within which various moveable components are disposed. In some embodiments, the housing 1305 includes or defines a plurality of tubes in which elements are slidably disposed and actuatable. A solid metal microneedle 1310 (not hollow core) is shown in the center of a tapered plastic introducer 1315. In various embodiments, one or more actuators such as actuator 1312 may be in mechanical communication or otherwise linked or connected to one or more components slidably disposed in the bore defined by 1315 or otherwise part of a treatment device that includes a plurality of the tubes such as those being actuated through various stages in FIG. 13. The introducer may include an electrical insulator. An injectable solution 1320 is shown relative to the needle 1310, introducer 1315, and piston 1325 in various stages with at least a portion of the fluid 1320 being delivered to or within tissue 1330 as shown. In various embodiments, the introducer 1315 is included for alignment and controlling movement of needle 1310. The piston 1325 includes an electrical insulator on top designed to be leak proof so that it can capture a quantity of injectable solution 1320 between the piston 1325 and the tapered plastic introducer 1315. Piston 1325 may define a channel or bore to secure needle and/or allow it to travel and move into and out of tissue 1330 under treatment within the housing 1305.

Stage B illustrates the insertion of one of the plurality of microneedles to a pre-determined depth between 0.1 and 5 mm in the patient's tissue 1330 (such as one or more layers of skin) or deeper in the patient subdermal fatty layer. In the following step, shown in Stage C, a first step of piston action plunges while capturing/sampling a fixed amount of injectable solution 1320 in the cylinder while pushing the tapered plastic introducer 1315 along the inserted solid needle 1310 to a predetermined depth in the skin. In various embodiments, the introducer and other components depicted may be slidably arranged or fit to support the various directions of travel and spatial translation shown. Stage D illustrates the retraction of the one of the plurality of microneedles from the skin to a position that enables or supports the delivery of injectable solution 1320 into the tapered plastic introducer 1315 while staying in contact with the injectable solution 1320.

In the next step, as shown in Stage E, a second step of piston action delivers injectable solution 1320 to a predetermined depth in the skin or deeper in the patient subdermal fatty layer or another target tissue layer of interest. The injectable solution 1320 modifies the electrical properties of the tissue in the vicinity of the tip of the tapered plastic introducer 1315. Stage F illustrates the delivery of RF power into the tissue with modified properties via the conductive conduit formed by the injectable solution remaining inside the tapered plastic introducer 1315. During the RF power delivery, the injectable solution forming the conductive conduit is in electrical contact with the one of the plurality of electrically conductive microneedles. The various elements and steps shown in FIG. 13 can be implemented in various device designs such as handpiece based or non-handpiece based designs for tissue treatment.

It will be appreciated that for clarity, this disclosure explicates various aspects of embodiments of the applicant's teachings, while omitting certain specific details wherever convenient or appropriate to do so. For example, discussion of like or analogous features in alternative embodiments may be somewhat abbreviated. Well-known ideas or concepts may also, for brevity, not be discussed in any great detail. The skilled person will recognize that some embodiments of the applicant's teachings may not require certain of the specifically described details in every implementation, which are set forth herein only to provide a thorough understanding of the embodiments. Similarly, it will be apparent that the described embodiments may be susceptible to alteration or variation according to common general knowledge without departing from the scope of the disclosure. The following detailed description of embodiments is not to be regarded as limiting the scope of the applicant's teachings in any manner.

The terms “about” and “substantially and “approximately” as used herein, refer to variations in a numerical quantity that can occur, for example, through measuring or handling procedures in the real world; through inadvertent error in these procedures; through differences/faults in the manufacture of electrical elements; through electrical losses; as well as variations that would be recognized by one skilled in the art as being equivalent so long as such variations do not encompass known values practiced by the prior art. Typically, the term “about” means greater or lesser than the value or range of values stated by 1/10 of the stated value, e.g., +10%. For instance, applying a voltage of about +3V DC to an element can mean a voltage between +2.7V DC and +3.3V DC. Likewise, wherein values are said to be “substantially identical,” the values may differ by up to 5%. Whether or not modified by the term “about” or “substantially” identical, quantitative values recited in the claims include equivalents to the recited values, e.g., variations in the numerical quantity of such values that can occur, but would be recognized to be equivalents by a person skilled in the art.

Moreover, nothing disclosed herein is intended to be dedicated to the public regardless of whether such disclosure is explicitly recited in the claims. To aid the Patent Office and any readers of any patent issued on this application in interpreting the claims appended hereto or otherwise presented throughout prosecution of this or any continuing patent application, applicant wishes to note that they do not intend any claimed feature to be construed under or otherwise to invoke the provisions of 35 USC 112(f), unless the phrase “means for” or “step for” is explicitly used in the particular claim.

All of the drawings submitted herewith include one or more ornamental features and views, each of which include solid lines any of which also incorporate and correspond to and provide support for dotted lines and alternatively, each of which include dotted lines any of which also incorporate and correspond to and provide support for solid lines.

The use of the terms “include,” “includes,” “including,” “have,” “has,” or “having” should be generally understood as open-ended and non-limiting unless specifically stated otherwise.

The use of the singular herein includes the plural (and vice versa) unless specifically stated otherwise. Moreover, the singular forms “a,” “an,” and “the” include plural forms unless the context clearly dictates otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions may be conducted simultaneously.

Where a range or list of values is provided, each intervening value between the upper and lower limits of that range or list of values is individually contemplated and is encompassed within the disclosure as if each value were specifically enumerated herein. In addition, smaller ranges between and including the upper and lower limits of a given range are contemplated and encompassed within the disclosure. The listing of exemplary values or ranges is not a disclaimer of other values or ranges between and including the upper and lower limits of a given range.

It should be appreciated that numerous changes can be made to the disclosed embodiments without departing from the scope of the present teachings. While the foregoing figures and examples refer to specific elements, this is intended to be by way of example and illustration only and not by way of limitation. It should be appreciated by the person skilled in the art that various changes can be made in form and details to the disclosed embodiments without departing from the scope of the teachings encompassed by the appended claims.

What is claimed is:

1. An apparatus for tissue treatment, comprising:
a base;
a plurality of hollow needles attached to the base, wherein each needle defines a hollow portion, at least one of the hollow portions of the plurality of hollow needles is in fluid communication with one or more fluid sources for delivering an electrically conductive fluid;
a guard that is slidable disposed relative to the base; and
an electrical interface configured to electrically couple the plurality of hollow needles to a control system, the electrical interface configured to receive one or more control signals from the control system, the control signal configured to manage at least one of (1) a temperature of fluid prior to delivery into at least one of the hollow portions and (2) a volume of fluid delivered into at least one of the hollow portions.

2. The apparatus of claim 1, wherein the guard is configured to protect the plurality of hollow needles, wherein the guard is configured to attach to a handpiece.

3. The apparatus of claim 1, wherein a portion of a surface of each needle comprises an electrically insulative coating.

4. The apparatus of claim 1 further comprising a microfluidic assembly, the microfluidic assembly defining a set of microfluidic channels that align with at least one of the hollow portions of the plurality of hollow needles to deliver the electrically conductive fluid therethrough and to an outlet of each needle.

5. The apparatus of claim 1 further comprising:
an energy source provided in communication with at least one of the needles, the energy configured to deliver thermal energy or RF energy relative to a tissue region.

6. The apparatus of claim 5, the apparatus further comprising:
the control system coupled to the electrical interface, the control system comprising a control module that varies characteristics of energy supplied by the energy source.

7. The apparatus of claim 5, wherein more than one of the needles are electrically conductive, and, wherein the energy source is configured to supply radio frequency electromagnetic energy to individual ones of the needles.

8. The apparatus of claim 7, further comprising balancing control of the electrical conductivity of the fluid to enable use of a relatively lower RF nominal power level and a relatively narrower RF impedance range than would be needed in the absence of balancing control of the electrical conductivity.

9. The apparatus of claim 7, further comprising balancing control of the temperature of the fluid prior to the fluid flowing into at least one of the hollow portions to enable use of a relatively lower RF nominal power level and a relatively narrower RF impedance range than would be needed in the absence of balancing control of fluid temperature.

10. The apparatus of claim 7, further comprising balancing control of at least one of (a) the electrical conductivity of the fluid and (b) the temperature of the fluid prior to flowing into at least one of the hollow portions to enable use of a relatively lower RF nominal power level and a relatively narrower RF impedance range than would be needed in the absence of balancing control of these.

11. The apparatus of claim 2, wherein the handpiece comprises at least one reservoir in fluid communication with at least one of the plurality of hollow needles, wherein the at least one reservoir comprises a deformable container configured to deliver reservoir contents into at least one of the hollow portions of the plurality of hollow needles in response to manual activation or a control signal from a control system, wherein the reservoir contents comprise an electrically conductive saline solution.

12. A skin treatment method comprising:
inserting a plurality of hollow needles into a dermal layer of skin, the plurality of hollow needles being attached to a base;
inducing a pattern of fractional tissue modification into a target tissue by delivering an electrically conductive fluid to the target tissue through two or more of the hollow portions of plurality of hollow needles; and
controlling at least one of (1) the temperature of an electrically conductive fluid prior to delivery into two or more of the hollow portions; (2) the electrical conductivity of an electrically conductive fluid delivered into two or more of the hollow portions and (3) an electrical parameter measured by a control system in response to delivery of an electrical signal and the electrically conductive fluid through one or more of the hollow needles, wherein electrically conductive properties of the fluid are suited to the tissue treatment; and removing the plurality of hollow needles from the dermal layer of skin.

13. The method of claim 12, wherein the electrically conductive fluid is configured to modify electrical conductivity of tissue.

14. The method of claim 12, further comprising heating the fluid, wherein the fluid thermally modifies a portion of the tissue or a tissue structure.

15. The method of claim 14, wherein the thermal modification to tissue comprises one or more of the following: denaturation, apoptosis, stimulation, coagulation, necrosis, charring, and ablation.

16. The method of claim 12, wherein the target tissue is one of dermal tissue, subdermal fat, subdermal connective tissue, and mucosal tissue.

17. The method of claim 12, wherein one or more of the plurality of hollow needles has a cut angle that ranges from about 20° to about 70°, wherein one or more of the plurality of hollow needles defines an outlet and has a notch to regulate fluid ingress, the notch disposed above the outlet.

18. The method of claim 12, wherein the electrically conductive fluid has an electrical conductivity suited to the target tissue.

19. The method of claim 12, wherein inducing a pattern of fractional tissue modification into a target tissue further comprises delivering RF energy into the target tissue after the electrically conductive fluid has been delivered therein.

20. An apparatus for tissue treatment comprising:
a housing comprising tissue contacting surface;
one or more actuators;
a plurality of elongate cylindrical tubes, wherein each tube defines a bore, wherein the plurality of tubes are disposed in the housing, wherein an opening of each tube is further defined by the tissue contacting surface; and
an electrical interface;
wherein each tube comprises:
an introducer disposed within the bore of the tube;
a solid needle, at least a portion of the needle surrounded by the introducer,
wherein the needle and introducer are slidably disposed within the tube, the one or more actuators configured to extend each needle from each bore such that each needle extends and retracts relative to the tissue contacting surface;
wherein the electrical interface is configured to electrically couple the needle and the one or more actuators to a control system, the interface providing signals to:
insert at least a portion of the needle into the tissue to create a tissue channel path;
insert at least a portion of the introducer into the tissue channel path;
retract the needle from the tissue channel path to separate a portion of the needle from a portion of the introducer disposed in the tissue channel path; and
introduce a fluid into the tissue channel path.
